(19) United States
(12) Patent Application Publication
(10) Pub. No.: US 2025/0320644 A1
(43) Pub. Date: Oct. 16, 2025

(54) HIGH LOFT NONWOVEN MATERIAL

(71) Applicant: DelStar Technologies, Inc., Alpharetta, GA (US)

(72) Inventors: Yasar Kiyak, Alpharetta, GA (US); Andrew G. Platt, Middletown, DE (US)

(21) Appl. No.: 19/173,950

(22) Filed: Apr. 9, 2025

Related U.S. Application Data
(60) Provisional application No. 63/634,519, filed on Apr. 16, 2024.

(51) Int. Cl. D04H 1/4382; A61F 13/0206; B01D 39/16; D04H 1/4374

(52) U.S. Cl. CPC D04H 1/43825; A61F 13/0206; B01D 39/1623; D04H 1/4374; D04H 1/43828; B01D 2239/0225; B01D 2239/0618; D10B 2505/04; D10B 2509/026

(57) ABSTRACT

Nonwoven materials are provided that comprise multicomponent and monocomponent fibers. The multicomponent fibers comprise first and second polymers and the monocomponent fibers comprise the second polymer or a third polymer. The second and/or third polymer has a higher melting temperature than the first polymer. The fibers are selected to provide increased loft to the materials and are suitable for a variety of different applications and products, such as filter media for air or liquid filters and absorbent pads for bandages, wound dressings, diapers, adult incontinence products, feminine hygiene products and the like. Absorbent pads are provided comprising high loft nonwoven material that increases the absorbency of the pad. Filter media is provided comprising high loft nonwoven material that increases the dust holding capacity of the media.

### CROSS-REFERENCE TO RELATED APPLICATIONS

This application claims the benefit of U.S. Provisional Application Ser. No. 63/634,519, filed Apr. 16, 2024, the complete disclosure of which is incorporated herein by reference.

### BACKGROUND

High loft or lofty nonwoven material is used in a wide variety of applications, such as absorbent pads used for bandages, wound dressings, hygiene-related products and diapers, and filter media used in, for example, air filters. High loft is a term used in textiles to describe a type of fabric that is characterized by its thickness and/or insulating properties because the volume of void space is greater than the volume of the total solid. In the case of personal care absorbent articles, such as feminine hygiene products and/or diapers, the fibers are typically processed and woven or knitted in a specific way to promote comfort (e.g., softness), surge management and fluid distribution to adjacent components of the article. For filter media, the high loft of the material may increase the overall dust holding capacity of the filter. For wound dressings or bandages, the high loft material increases the absorbency of the article.

In air through bonded carded nonwoven fibers, the loftiness of a substrate can be controlled by various means known to those of skill in the art. For example, loftiness can be increased by applying less compression force onto the media during bonding. In another example, a high loft nonwoven material can be manufactured with fibers having larger linear densities, which is the measure of the fiber's mass per unit length or length per unit mass.

In certain applications, high loft nonwoven material may comprise spunbond fibers having crimped multicomponent fibers. The multicomponent fibers are air through bonded, which increases the overall loft of the material.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

Nonwoven materials are provided that comprise both multicomponent and monocomponent fibers. The fibers are selected to provide increased loft to the materials and are suitable for a variety of different applications and products, such as filter media for air or liquid filters and absorbent pads for bandages, wound dressings, diapers, adult incontinence products, feminine hygiene products, pleated material, such as coffee filters, water filters, tea bags, pleated air filters and the like.

In one aspect, a nonwoven material comprises multicomponent fibers prepared from first and second polymers and a monocomponent fibers prepared from a third polymer. The second and third polymers have a higher melting temperature than the first polymer.

In one embodiment, the second polymer is the same as the third polymer. In another embodiment, the second polymer is different from the third polymer.

In various embodiments, the melting temperatures of the first and second polymers are selected such that the first polymer substantially melts during the heat bonding process, but the second polymer does not substantially melt. The melting temperature of the second polymer is at least about 5° C., or at least about 15° C. higher than the melting temperature of the first polymer. The monocomponent fibers increase the loftiness, softness, and void spaces for air passage in the material. In addition, the monocomponent fibers positioned within the depth of the media increase the overall compression strength of the material.

In various embodiments, the monocomponent fibers comprise about 10% to about 50% by weight of the material. The multicomponent fibers may comprise about 50% to about 90% by weight of the material. In an exemplary embodiment, the multicomponent fibers comprise about 60% to about 80%, or about 70%, by weight of the material and the monocomponent fibers comprise about 20% to about 40%, or about 30%, by weight of the material.

In various embodiments, the material comprises first and second layers. In one such embodiment, the first layer comprises the multicomponent fibers and the second layer comprises the monocomponent fibers. In another embodiment, the first and second layers each comprise the monocomponent fibers and the multicomponent fibers. In another embodiment, the monocomponent fibers and the multicomponent fibers alternate with each other along the width or length of the material in each of the first and second layers.

The multicomponent fibers may have any suitable configuration such as concentric core/sheath, eccentric core/sheath, side by side, segmented pie, segmented cross, segmented ribbon, island in the sea, hollow bicomponent fiber. hollow segmented pic, trilobal, tipped multilobal, mixed fibers, striped fibers, conductive fibers, and combinations thereof. In an exemplary embodiment, the multicomponent fibers comprise bicomponent fibers having either a side by side or a core/sheath configuration. The core may be concentric or eccentric with the sheath.

In one such embodiment, the multicomponent fibers have a core/sheath configuration with the core comprising the second polymer and the sheath comprising the first polymer. Thus, the sheath substantially melts during the bonding process, while the core remains substantially unmelted. The sheath may comprise at least about 30% by weight, or at least about 50% by weight, or at least about 60% by weight, or about 70% to about 90% by weight, or about 70% by weight of the bicomponent fiber.

The first polymer may comprise any suitable material including, but not limited to, thermoplastic liquid crystalline polymers, polyesters, co-polyesters, polyethylene terephthalate (PET), low melting polylactic acid (PLA), polyethylene (PE), high density polyethylene (HDPE), low melting polyethylene terephthalate (CoPET), polypropylene (PP), polybutylene terephthalate (PBT), polytrimethylene terephthalate (PTT), polyamides, polyolefins, and combinations thereof. In an exemplary embodiment, the first polymer comprises HDPE.

The second polymer may comprise any suitable material including, but not limited to, thermoplastic liquid crystalline polymers, polyesters, co-polyesters, polyethylene terephthalate (PET), polylactic acid (PLA), polyethylene (PE), high density polyethylene (HDPE), polyethylene terephthalate (CoPET), polypropylene (PP), polybutylene terephthalate (PBT), polytrimethylene terephthalate (PTT), polyamides, polyolefins, and combinations thereof. In an exemplary embodiment, the second polymer comprises PET.

In one embodiment, the first and second polymers are selected from the same material having different melting points (e.g., different tacticity). For example, the first and second polymers may both comprise a different PP material.

The fibers may be manufactured by any suitable method, including, without limitation, meltblown, bicomponent meltblown, spunbond or spunlace, bicomponent spunbond, heat-bonded, carded, air-through bonded carded, air-laid, wet-laid, extrusion, co-formed, needlepunched, stitched, hydraulically entangled or the like. In an exemplary embodiment, the fibers are spunbond.

The fibers may have any suitable cross-sectional shape, such as circular, oval, rectangular, square, triangular, multilobal, and the like. The fibers may have a thickness of about 5 to 1000 microns, or about 100 microns or less, or about 20 microns to about 40 microns. The fibers may have a basis weight of about 5 gsm to about 300 gsm or about 20 gsm to about 60 gsm, or about 40 gsm.

In various embodiments, the fibers may include additives such as other polymers, nucleating agents, plasticizer, slip additives, elastomeric polymers and the like.

In another aspect, an absorbent pad is provided comprising the nonwoven material described above. In certain aspects, a product, such as a wound dressing, bandage (e.g., a finger bandage), diaper and/or feminine hygiene product is provide comprising an absorbent pad with the nonwoven material described above.

In another aspect, a filter media and a filter are provided comprising the nonwoven material described above.

In another aspect, a nonwoven material comprises multicomponent fibers prepared from first and second polymers and monocomponent fibers prepared from the second polymer. The multicomponent fibers comprise about 50% to about 90% by weight of the nonwoven material.

In various embodiments, the monocomponent fibers comprise about 10% to about 50% by weight of the material. In an exemplary embodiment, the multicomponent fibers comprise about 60% to about 80%, or about 70%, by weight of the material and the monocomponent fibers comprise about 20% to about 40%, or about 30%, by weight of the material.

In various embodiments, the first polymer comprises at least about 30% by weight, or at least about 50% by weight, or at least about 60% by weight or about 70% to about 90% by weight, or about 70% by weight of the multicomponent fiber.

The second polymer may have a higher melting temperature than the first polymer. For example, the melting temperature of the second polymer may be at least about 5° C., or at least about 15° C. higher than a melting temperature of the first polymer

In another aspect, a nonwoven material comprises multicomponent fibers comprising first and second polymers and monocomponent fibers comprising a third polymer. The second and third polymers have a higher melting temperature than the first polymer. For example, the melting temperature of the second and third polymers may be at least about 5° C., or at least about 15° C. higher than a melting temperature of the first polymer

In one embodiment, the third polymer comprises a different material than the second polymer. In another embodiment, the third polymer comprises the same material as the second polymer.

It is to be understood that both the foregoing general description and the following detailed description are exemplary and explanatory only and are not restrictive. Additional features will be set forth in part in the description which follows or may be learned by practice of the description.

### BRIEF DESCRIPTION OF THE DRAWINGS

The accompanying drawings, which are incorporated in and constitute a part of this specification, illustrate several embodiments, and serve to explain the principles herein.

FIG. 1 is a schematic illustration of a nonwoven material comprising first and second polymers.

FIG. 2 is a schematic illustration of another embodiment of a nonwoven material comprising first and second polymers;

FIG. 3 is a schematic illustration of another embodiment of a nonwoven material comprising first and second polymers;

FIG. 4 is a schematic illustration of another embodiment of a nonwoven material comprising first and second polymers;

FIG. 5 is a schematic illustration of another embodiment of a nonwoven material comprising first and second polymers;

FIG. 6 is a schematic illustration of another embodiment of a nonwoven material comprising first and second polymers;

FIG. 7 is a perspective top view of a bandage having an adhesive layer and an absorbent pad;

FIG. 8 is a cross-sectional view of the bandage of FIG. 7; and

FIG. 9 is a perspective view of a filter.

### DETAILED DESCRIPTION

This description and the accompanying drawings illustrate exemplary embodiments and should not be taken as limiting, with the claims defining the scope of the description, including equivalents. Various mechanical, compositional, structural, and operational changes may be made without departing from the scope of this description and the claims, including equivalents. In some instances, well-known structures and techniques have not been shown or described in detail so as not to obscure the description. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

High loft nonwoven materials are provided that comprise multicomponent and monocomponent fibers. The fibers are selected to provide increased loft to the materials and are suitable for a variety of different applications and products, such as filter media for air or liquid filters and absorbent pads for bandages, wound dressings, diapers, adult incontinence products, feminine hygiene products, pleated material, such as coffee filters, water filters, tea bags, pleated air filters and the like. As used herein, the term “high loft” means that the volume of void space within the material is greater than the volume of the total solid.

In certain applications, the nonwoven materials discussed herein may be included as part of a filter device that traps or absorbs contaminants, such as a liquid filter, a gas filter for home and commercial air filtration (e.g., HVAC), a surgical mask, or other face covering, CPAP filters, vacuum bags, gas turbine and compressor air intake filters, panel filters or the like. The filter device may be a mechanical filter, absorption filter, sequestration filter, ion exchange filter, reverse osmosis filter, surface filter, depth filter or the like, and may be designed to remove many different types of contaminants from the air, water, or others.

In other applications, the nonwoven material described herein is particularly suitable for use in absorbent pads for bandages, wound dressings, diapers, adult incontinence products, feminine hygiene products and the like. Although it should be understood that the terms “absorbent pad” and “pad” as used herein do not restrict the purpose of the absorbent article to which they refer, to mere padding, serving simply as a cushioning or stuffing between other layers. In fact, the term may instead be directed broadly to a material which is thin, flat, and comprising fibers, such as the absorbent layer of a bandage.

In other applications, the nonwoven material may be used in materials where pleating is required, such as coffee filters, tea bags, water filters, air filters and the like. The nonwoven material provides pleat stability and thermal stability and may provide a structure that is processable at high throughput with limited shrinkage.

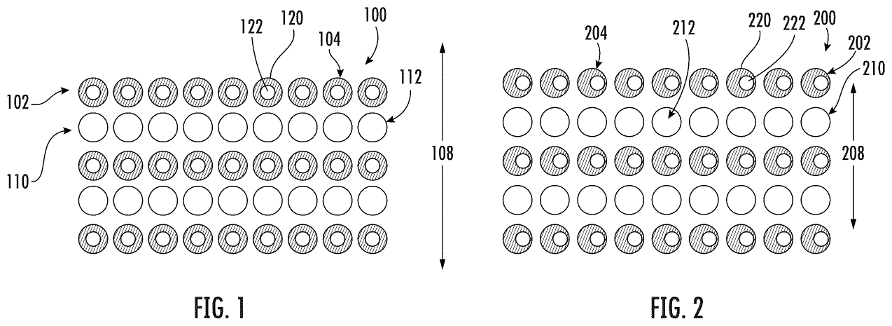

FIG. 1 schematically illustrates one embodiment of a nonwoven material 100, which may comprise a substrate, sheet, layer, film, web, or other media comprising fibers. The nonwoven material 100 may comprise a structure of individual fibers or threads that are interlaid, interlocked, or bonded together. Nonwoven fabrics may include sheets or web structures bonded together by entangling fiber or filaments (and by perforating films) mechanically, thermally, or chemically. They may be substantially flat, porous sheets that are made directly from separate fibers or molten plastic or plastic film.

As shown, nonwoven material 100 comprises a first row 102 of multicomponent fibers 104 and a second row 110 of monocomponent fibers 112. The material 100 may comprise successive alternating rows of multicomponent 104 and monocomponent fibers 112 along a thickness 108 of material 100. The fibers may include biocomponent fibers that include two or more different filaments bonded to each other.

In this embodiment, multicomponent fibers 104 comprise a bicomponent fiber having a core/sheath configuration with a first polymer comprising the sheath 120 and a second polymer comprising the core 122. In certain embodiments, the monocomponent fibers 104 also comprise the second polymer. In other embodiments, monocomponent fibers 104 comprise a third polymer. In this embodiment, multicomponent fibers 104 comprise a concentric core/sheath configuration, although it is contemplated that the core may be eccentric with the sheath. The sheath may comprise at least about 30% by weight, or at least about 50% by weight, or about 70% to about 90%, or about 70% by weight of the multicomponent fiber 104.

The melting temperatures of the first and second polymers are selected such that the first polymer substantially melts during the heat bonding process, but the second polymer does not substantially melt. In embodiments, wherein monocomponent fibers 104 comprise a third polymer, the melting temperature of the third polymer is selected such that it does not substantially melt during the heat bonding process. Thus, the melting temperature of the second and third polymers is at least about 5° C., or at least about 15° C. higher than a melting temperature of the first polymer. This increases the loftiness, softness, and void spaces for air passage in the material. In addition, providing monocomponent fibers 112 having higher melting temperatures within the depth of the material 100 increase the overall compression strength of material 100.

In various embodiments, monocomponent fibers 112 comprise about 10% to about 50% by weight of the material. Multicomponent fibers 104 comprise about 50% to about 90% by weight of the material. In an exemplary embodiment, multicomponent fibers 104 comprise about 60% to about 80% or about 70% by weight of the material and monocomponent fibers 112 comprise about 20% to about 40% or about 30% by weight of the material.

It has been found that using multicomponent fibers with different shrinkage characteristics further increases the loftiness of the material. For example, multicomponent fibers having a first polymer with a lower melting temperature than a second polymer increases the shrinkage characteristics of the first polymer relative to the second polymer (i.e., the second polymer does not fully melt during the heat bonding process). In some cases, special additives, such as an elastomer, may be added to augment the shrinkage traits of the first polymer.

The first polymer may comprise any suitable material including, but not limited to, thermoplastic liquid crystalline polymers, polyesters, co-polyesters, polyethylene terephthalate (PET), low melting polylactic acid (PLA), polyethylene (PE), high density polyethylene (HDPE), low melting polyethylene terephthalate (CoPET), polypropylene (PP), polybutylene terephthalate (PBT), polytrimethylene terephthalate (PTT), polyamides, polyolefins, and combinations thereof. Other conventional fiber materials are contemplated. In an exemplary embodiment, the first polymer comprises HDPE.

The second polymer may comprise any suitable material including, but not limited to, thermoplastic liquid crystalline polymers, polyesters, co-polyesters, polyethylene terephthalate (PET), polylactic acid (PLA), polyethylene (PE), high density polyethylene (HDPE), polyethylene terephthalate (CoPET), polypropylene (PP), polybutylene terephthalate (PBT), polytrimethylene terephthalate (PTT), polyamides, polyolefins, and combinations thereof. Other conventional fiber materials are contemplated. In an exemplary embodiment, the second polymer comprises PET.

In certain embodiments, the first and second polymers are selected from the same material having different melting points (e.g., different tacticity). For example, the first and second polymers may both comprise a different PP material.

The fibers may be manufactured by any suitable method, including, without limitation, meltblown, bicomponent meltblown, spunbond or spunlace, bicomponent spunbond, heat-bonded, carded, air-through bonded carded, air-laid, wet-laid, extrusion, co-formed, needlepunched, stitched, hydraulically entangled or the like.

In an exemplary embodiment, the fibers comprise continuous spunbond fibers forming by meltspinning. Spunbond media is generally more cost effective than other manufacturing methods because it involves a relatively high throughput of the fibers. The multicomponent fibers may be formed by extruding two or more polymers from the same spinneret or spin pack with both polymers contained within the same filament. The filaments are then drawn for increasing orientation, and collected onto a conveyor belt. The monocomponent fiber may be formed through standard spunbond manufacturing techniques. Both the monocomponent and biocomponent fibers may be calendared together after they pass through the spin packs. The bicomponent spunbond fibers may be manufactured, for example, according to the systems and methods described in U.S. Pat. No. 7,981,226, the complete disclosure of which is incorporated herein by reference for all purposes.

The monocomponent and multicomponent fibers may be formed into a nonwoven material by a combination of heat and pressure. The heating may be applied through any suitable technique known in the art. In an exemplary embodiment, the fibers are heated by air through bonding and ultrasonic welding. The final nonwoven material has higher loft because of the nonbondable (i.e., higher melting temperature second polymer) that creates void spaces in the structure.

The fibers contemplated may have any suitable shape in cross-section, including without limitation, circular, kidney bean, dog bone, trilobal, barbell, bowtie, star, Y-shaped, triangular, multilobal, square, oval and others. These shapes and/or other conventional shapes may be used with any of the embodiments described herein to obtain the desired performance characteristics.

The fibers may have thicknesses that are suitable for the application. In some embodiments, the fibers have at least one dimension (e.g., a diameter in the case of circular cross-sectional fibers) in the range of about 1 to about 10,000 microns or about 1 to about 1,000 microns or about 10 to 100 micrometers, or about 20 microns to about 40 microns. The fibers preferably have a linear density of less than about 15 denier, or less than about 10 denier or below 9 denier.

The fibers may have a basis weight of about 5 gsm to about 300 gsm or about 20 gsm to about 60 gsm, or about 40 gsm.

In various embodiments, the fibers may include additives such as other polymers, nucleating agents, plasticizer, slip additives, elastomeric polymers and the like.

Referring now to FIG. 2, another embodiment of a nonwoven material 200 comprises a first row 202 of multicomponent fibers 204 and a second row 210 of monocomponent fibers 212. The material 200 may comprise successive alternating rows of multicomponent 204 and monocomponent fibers 212 along a thickness 208 of material 200. Multicomponent fibers 204 comprise a core/sheath configuration with a first polymer comprising the sheath 220 and a second polymer comprising the core 222. The monocomponent fibers 204 also comprise the second polymer. In this embodiment, multicomponent fibers 104 comprise an eccentric core/sheath configuration. The sheath may comprise at least about 30% by weight, or at least about 50% by weight, or about 70% to about 90%, or about 70% by weight of the multicomponent fiber 204. The eccentric core of the multicomponent fibers further increases the loftiness of material 200.

The melting temperatures of the first and second polymers are selected such that the first polymer substantially melts during the heat bonding process, but the second polymer does not substantially melt. The melting temperature of the second polymer is at least about 5° C., or at least about 15° C. higher than a melting temperature of the first polymer.

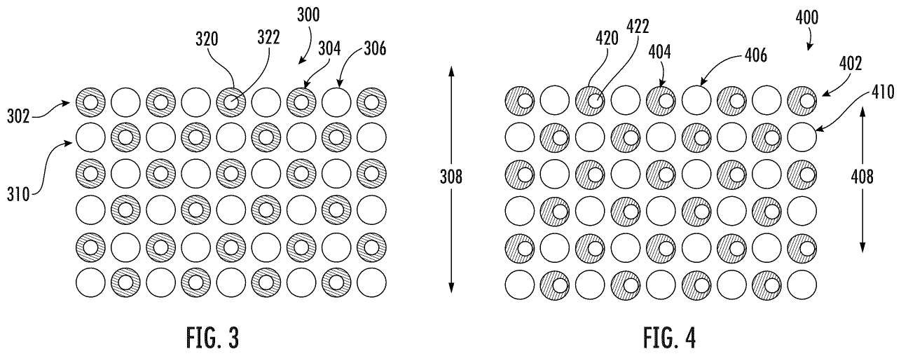

Referring now to FIG. 3, another embodiment of a nonwoven material 300 comprises a first row 302 of both multicomponent fibers 304 and monocomponent fibers 306. In this embodiment, multicomponent and monocomponent fibers 304, 306 alternate with each other along the length or width of first row 302. Nonwoven material 300 includes a second row 310 of both multicomponent fibers 304 and monocomponent fibers 306 that alternate with each other along the length or width of second row 310. Material 300 may include successive rows of alternating monocomponent and multicomponent fibers along a thickness 308 of material 300.

Multicomponent fibers 304 comprise a core/sheath configuration with a first polymer comprising the sheath 320 and a second polymer comprising the core 322. The monocomponent fibers 304 also comprise the second polymer. In this embodiment, multicomponent fibers 304 comprise a concentric core/sheath configuration, although it is contemplated that the core may be eccentric with the sheath. The sheath may comprise at least about 30% by weight, or at least about 50% by weight, or about 70% to about 90%, or about 70% by weight of the multicomponent fiber 304.

In an exemplary embodiment, each successive row in material 300 also alternates with the rows above and below that particular row. Thus, for example, a monocomponent fiber 306 in the first row 302 is positioned above a multicomponent fiber 304 in second row 310. Similarly, a multicomponent fiber 304 in first row 302 is positioned above a monocomponent fiber 306 in second row 310. Of course, other configurations are contemplated. For example, the monocomponent and multicomponent fibers may have different configurations within each row (e.g., two monocomponent fibers and then a multicomponent fiber, or vice versa). In addition, the rows may have different configurations, e.g., the multicomponent and/or monocomponent fibers may be aligned with each other in each row, or each row may have a different sequence of multicomponent and monocomponent fibers.

Referring now to FIG. 4, another embodiment of a nonwoven material 400 comprises a first row 402 of both multicomponent fibers 404 and monocomponent fibers 406. In this embodiment, multicomponent and monocomponent fibers 404, 406 alternate with each other along the length or width of first row 402. Nonwoven material 400 includes a second row 410 of both multicomponent fibers 404 and monocomponent fibers 406 that alternate with each other along the length or width of second row 410. Material 400 may include successive rows of alternating monocomponent and multicomponent fibers along a thickness 408 of material.

Multicomponent fibers 404 comprise a core/sheath configuration with a first polymer comprising the sheath 420 and a second polymer comprising the core 422. The monocomponent fibers 406 also comprise the second polymer. In this embodiment, multicomponent fibers 404 comprise an eccentric core/sheath configuration. The sheath may comprise at least about 30% by weight, or at least about 50% by weight, or about 70% to about 90%, or about 70% by weight of the multicomponent fiber 404.

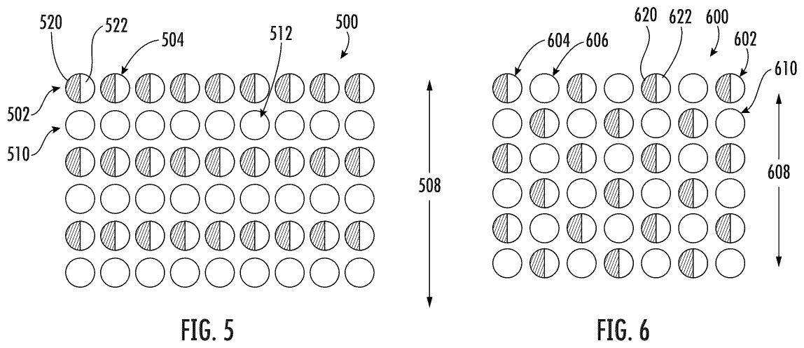

Referring now to FIG. 5, another embodiment of a nonwoven material 500 comprises comprises a first row 502 of multicomponent fibers 504 and a second row 510 of monocomponent fibers 512. The material 500 may comprise successive alternating rows of multicomponent 504 and monocomponent fibers 512 along a thickness 508 of material 500. Multicomponent fibers 504 comprise a side by side configuration with a first polymer comprising a first side 520 and a second polymer comprising a second side 522 The monocomponent fibers 512 also comprise the second polymer.

Referring now to FIG. 6, another embodiment of a nonwoven material 600 comprises a first row 602 of both multicomponent fibers 604 and monocomponent fibers 606. In this embodiment, multicomponent and monocomponent fibers 604, 606 alternate with each other along the length or width of first row 602. Nonwoven material 600 includes a second row 610 of both multicomponent fibers 604 and monocomponent fibers 606 that alternate with each other along the length or width of second row 610. Material 600 may include successive rows of alternating monocomponent and multicomponent fibers along a thickness 608 of material.

Multicomponent fibers 604 comprise a side by side configuration with a first polymer comprising a first side 620 and a second polymer comprising a second side 622 The monocomponent fibers 606 also comprise the second polymer.

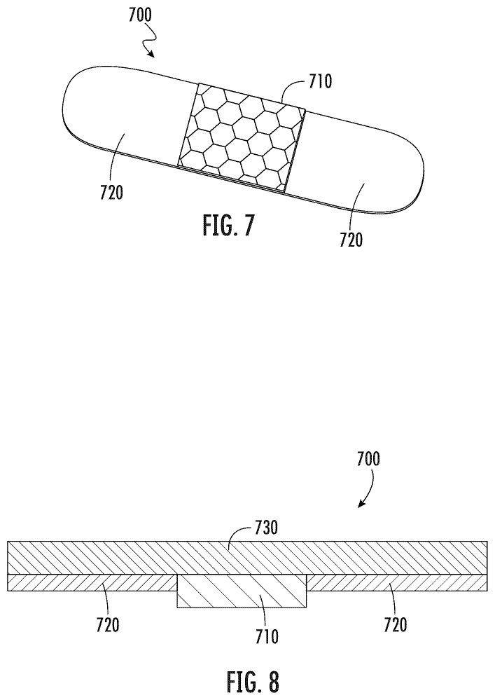

Referring now to FIGS. 7 and 8, an illustrative bandage 700 comprises an adhesive layer 720 and an absorbent pad 710. Absorbent pad 710 comprises one or more of the woven materials described above. The additional loft provided by woven materials increases the absorbency of the pad 710.

An additional layer, such as a backing layer 730, may also be a layer of bandage 700. Bandage 700 may comprise one or more additional layers (not shown), or more than one layer of any shown in FIGS. 7 and 8. In certain embodiments, a wound-release layer (not shown) may be added to the surface of absorbent pad 710 that contacts the wound. For example, a porous net may be placed between the wound and absorbent pad 710 to facilitate removal of the pad and/or bandage. It is understood that other types of backing or release layers may be added, and may include layers associated with easy removal of the pad or bandage from its packaging. Alternatively, a release layer may be present on the surface of adhesive layer 8 such as, for example, silicone paper release strips (not shown), which the user may remove before placement of the bandage onto skin/

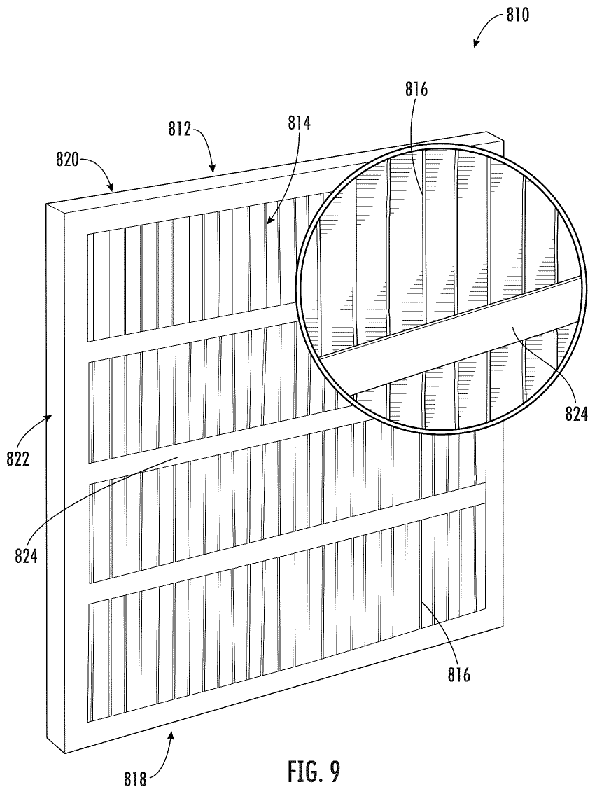

Referring now to FIG. 9, air filter 810 according to one embodiment generally comprises an outer frame 812 and a filter media 814. Filter media 814 comprises one or more of the woven materials described above. The additional loft provided by woven materials increases the overall dust holding capacity of the filter media 814. In some embodiments, the filter media may be scored, pleated, or folded into a pleated filter. The pleats may be formed by various conventional pleating operations that include, but are not limited to, bar, rotary, and star gear pleating operations.

In the representative embodiment, filter media 810 may comprises a plurality of pleats 816 extending in a substantially linear direction from a first end 818 of frame 812 to a second end 820. Frame 812 may comprise any standard frame used in self-supporting air filters, such as a HEPA filter, and generally includes an outer support 822 that surrounds filter media 814 and a plurality of horizontal support bars 824 that extend across filter media 814. Frame 812 may be suitable coupled to filter media 814 with conventional adhesives.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims.

For example, in a first aspect, a first embodiment is a nonwoven material comprising multicomponent fibers prepared from first and second polymers and monocomponent fibers prepared from a third polymer. The second and third polymers have a higher melting temperature than the first polymer.

A second embodiment is the first embodiment, wherein the second polymer is the same as the third polymer.

A third embodiment is any combination of the first two embodiments, wherein the second polymer is different from the third polymer.

A 4th embodiment is any combination of the first 3 embodiments, wherein a melting temperature of the second polymer is at least about 5 degrees Celsius higher than a melting temperature of the first polymer.

A 5th embodiment is any combination of the first 4 embodiments, wherein a melting temperature of the second polymer is at least about 15 degrees Celsius higher than a melting temperature of the first polymer.

A 6th embodiment is any combination of the first 5 embodiments, wherein the multicomponent fibers comprise about 50% to about 90% by weight of the material.

A 7th embodiment is any combination of the first 6 embodiments, wherein the monocomponent fibers comprise about 10% to about 50% by weight of the material.

An 8th embodiment is any combination of the first 7 embodiments, wherein the multicomponent fibers comprise about 70% by weight of the material and the monocomponent fibers comprise about 30% by weight of the material.

A 9th embodiment is any combination of the first 8 embodiments, wherein the material comprises first and second layers.

A 10th embodiment is any combination of the first 9 embodiments, wherein the first layer comprises the multicomponent fibers and the second layer comprises the monocomponent fibers.

An 11th embodiment is any combination of the first 10 embodiments, wherein the first and second layers each comprise the monocomponent fibers and the multicomponent fibers.

A 12th embodiment is any combination of the first 10 embodiments, wherein the monocomponent fibers and the multicomponent fibers alternate with each other in each of the first and second layers.

A 13th embodiment is any combination of the first 12 embodiments, wherein the multicomponent fibers have a configuration selected from the group consisting of concentric core/sheath, eccentric core/sheath, side by side, segmented pie, segmented cross, segmented ribbon, island in the sea, hollow bicomponent fiber. hollow segmented pie, trilobal, tipped multilobal, mixed fibers, striped fibers, conductive fibers, and combinations thereof.

A 14th embodiment is any combination of the first 13 embodiments, wherein the multicomponent fibers have a core/sheath configuration.

A 15th embodiment is any combination of the first 14 embodiments, wherein the core comprises the second polymer and the sheath comprises the first polymer.

A 16th embodiment is any combination of the first 15 embodiments, wherein the sheath comprises at least about 50% by weight of the multicomponent fiber.

A 17th embodiment is any combination of the first 16 embodiments, wherein the sheath comprises about 70% by weight of the multicomponent fiber.

An 18th embodiment is any combination of the first 17 embodiments, wherein the core is concentric.

A 19th embodiment is any combination of the first 18 embodiments, wherein the core is eccentric.

A 20th embodiment is any combination of the first 19 embodiments, wherein the multicomponent fibers have a side by side configuration.

A 21st embodiment is any combination of the first 20-embodiments, wherein the first polymer is selected from the group consisting of thermoplastic liquid crystalline polymers, polyesters, co-polyesters, polyethylene terephthalate (PET), low melting polylactic acid (PLA), polyethylene (PE), high density polyethylene (HDPE), low melting polyethylene terephthalate (CoPET), polypropylene (PP), polybutylene terephthalate (PBT), polytrimethylene terephthalate (PTT), polyamides, polyolefins, and combinations thereof.

A 22nd embodiment is any combination of the first 21 embodiments, wherein the first polymer comprises HDPE.

A 23rd embodiment is any combination of the first 22 embodiments, wherein the second polymer is selected from the group consisting of thermoplastic liquid crystalline polymers, polyesters, co-polyesters, polyethylene terephthalate (PET), polylactic acid (PLA), polyethylene (PE), high density polyethylene (HDPE), polyethylene terephthalate (CoPET), polypropylene (PP), polybutylene terephthalate (PBT), polytrimethylene terephthalate (PTT), polyamides, polyolefins, and combinations thereof.

A 24th embodiment is any combination of the first 23 embodiments, wherein the second polymer comprises PET.

A 25th embodiment is any combination of the first 24 embodiments, wherein the monocomponent and multicomponent fibers are spunbond.

A 26th embodiment is any combination of the first 25 embodiments, wherein the monocomponent and multicomponent fibers have a diameter of about 100 microns or less.

A 27th embodiment is any combination of the first 26 embodiments, wherein the diameter is about 20 microns to about 40 microns.

A 28th embodiment is any combination of the first 27 embodiments, wherein the material has a basis weight of about 5 gsm to about 300 gsm.

A 29th embodiment is any combination of the first 28 embodiments, wherein the basis weight is about 20 gsm to about 60 gsm.

In another aspect, an absorbent pad is provided comprising the nonwoven material of any of the above 29 embodiments.

In another aspect, a wound dressing is provided comprising the absorbent pad.

In another aspect, a bandage is provided comprising the absorbent pad.

In another aspect, a diaper is provided comprising the absorbent pad.

In another aspect, a feminine hygiene product is provided comprising the absorbent pad.

In another aspect, a filter media is provided comprising the nonwoven material of any of the above 28 embodiments.

In another aspect, a filter is provided comprising the filter media.

In another aspect, a first embodiment is a nonwoven material comprising multicomponent fibers prepared from first and second polymers and monocomponent fibers prepared from the second polymer. The multicomponent fibers comprise about 50% to about 90% by weight of the nonwoven material.

A second embodiment is the first embodiment, wherein the monocomponent fibers comprise about 10% to about 50% by weight of the material.

A 3rd embodiment is any combination of the first two embodiments, wherein the multicomponent fibers comprise about 70% by weight of the material and the monocomponent fibers comprise about 30% by weight of the material.

A 4th embodiment is any combination of the first 3 embodiments, wherein the first polymer comprises at least about 50% by weight of the multicomponent fiber.

A 5th embodiment is any combination of the first 4 embodiments, wherein the first polymer comprises about 70% by weight of the multicomponent fiber.

A 6th embodiment is any combination of the first 5 embodiments, wherein the second polymer has a higher melting temperature than the first polymer.

A 7th embodiment is any combination of the first 6 embodiments, wherein a melting temperature of the second polymer is at least about 5 degrees Celsius higher than a melting temperature of the first polymer.

An 8th embodiment is any combination of the first 7 embodiments, wherein a melting temperature of the second polymer is at least about 15 degrees Celsius higher than a melting temperature of the first polymer.

A 9th embodiment is any combination of the first 3 embodiments,

In another aspect, an absorbent pad is provided comprising the nonwoven material of any of the above 9 embodiments.

In another aspect, a wound dressing is provided comprising the absorbent pad.

In another aspect, a bandage is provided comprising the absorbent pad.

In another aspect, a diaper is provided comprising the absorbent pad.

In another aspect, a feminine hygiene product is provided comprising the absorbent pad.

In another aspect, a filter media is provided comprising the nonwoven material of any of the above 9 embodiments.

In another aspect, a filter is provided comprising the filter media.

What is claimed is:

1. A nonwoven material comprising:
multicomponent fibers prepared from first and second polymers; and
monocomponent fibers prepared from a third polymer, wherein the second and third polymers have a higher melting temperature than the first polymer.

2. The nonwoven material of claim 1, wherein the third polymer is the same as the second polymer.

3. The nonwoven material of claim 1, wherein the third polymer is different from the second polymer.

4. The nonwoven material of claim 1, wherein a melting temperature of the second and third polymers is at least about 5 degrees Celsius higher than a melting temperature of the first polymer.

5. The nonwoven material of claim 1, wherein a melting temperature of the second and third polymers is at least about 15 degrees Celsius higher than a melting temperature of the first polymer.

6. The nonwoven material of claim 1, wherein the multicomponent fibers comprise about 50% to about 90% by weight of the material and the monocomponent fibers comprise about 10% to about 50% by weight of the material.

7. The nonwoven material of claim 1, wherein the material comprises first and second layers.

8. The nonwoven material of claim 7, wherein the first layer comprises the multicomponent fibers and the second layer comprises the monocomponent fibers.

9. The nonwoven material of claim 7, the first and second layers each comprise the monocomponent fibers and the multicomponent fibers.

10. The nonwoven material of claim 7, wherein the monocomponent fibers and the multicomponent fibers alternate with each other in each of the first and second layers.

11. The nonwoven material of claim 1, wherein the multicomponent fibers have a configuration selected from the group consisting of concentric core/sheath, eccentric core/sheath, side by side, segmented pie, segmented cross, segmented ribbon, island in the sea, hollow bicomponent fiber. hollow segmented pie, trilobal, tipped multilobal, mixed fibers, striped fibers, conductive fibers, and combinations thereof.

12. The nonwoven material of claim 1, wherein the multicomponent fibers have a core/sheath configuration.

13. The nonwoven material of claim 12, wherein the core comprises the second polymer and the sheath comprises the first polymer.

14. The nonwoven material of claim 12, wherein the sheath comprises at least about 50% by weight of the multicomponent fiber.

15. The nonwoven material of claim 1, wherein the multicomponent fibers have a side by side configuration.

16. An absorbent pad comprising the nonwoven material of claim 1.

17. A filter media comprising the nonwoven material of claim 1.

18. A nonwoven material comprising:
multicomponent fibers prepared from first and second polymers; and
monocomponent fibers prepared from the second polymer, wherein the multicomponent fibers comprise about 50% to about 90% by weight of the nonwoven material.

19. The nonwoven material of claim 18, wherein the monocomponent fibers comprise about 10% to about 50% by weight of the material.

20. The nonwoven material of claim 18, wherein the first polymer comprises at least about 50% by weight of the multicomponent fiber.

21. The nonwoven material of claim 18, wherein the second polymer has a higher melting temperature than the first polymer.
